(19) United States
(12) Patent Application Publication
(10) Pub. No.: US 2009/0093838 A1
(43) Pub. Date: Apr. 9, 2009

(54) SHAPE MEMORY INTRAGASTRIC BALLOON

(75) Inventor: Pascal Paganon, Serezin du Rhone (FR)

Correspondence Address: BRYAN CAVE POWELL GOLDSTEIN, ONE ATLANTIC CENTER FOURTEENTH FLOOR, 1201 WEST PEACHTREE STREET NW, ATLANTA, GA, 30309-3488, US

(73) Assignee: Compagnie Europeenne d'Elude et de Dispositifs pour L'lmplantation par Laparoscopie, Vienne (FR)

(21) Appl. No.: 12/279,759

(22) PCT Filed: Feb. 16, 2007
(86) PCT No.: PCT/FR07/00285

§ 371 Date: Dec. 11, 2008

(30) Foreign Application Priority Data
Feb. 17, 2006 (FR) 0601462

(51) Int. Cl. A61M 29/02

(52) U.S. Cl. 606/192; 29/527.1

(57) ABSTRACT

An expandable intragastric balloon (1) to be implanted in, and subsequently removed from, the stomach (2) of a patient through natural orifices as part of a treatment for obesity. The intragastric balloon (1) includes an outer casing (3) sufficiently flexible that the outer casing can change from a first configuration, the introduction configuration, to a second configuration, the deployed configuration, in which the balloon adopts a functional shape. The balloon (1) includes means for configuring the balloon in an extraction configuration (10), the means exerting a stress on the intragastric balloon (1) such that the balloon changes from the deployed configuration to a third noticeably-smaller contracted volume configuration, the extraction configuration, by acting on the outer casing (3) such that the casing (3) essentially returns to or towards a predetermined shape corresponding to the extraction configuration.

### PRIORITY CLAIM

This patent application is a U.S. National Phase of International Patent Application No. PCT/FR2007/000285, filed Feb. 16, 2007, which claims priority to French Patent Application No. 0601462, filed Feb. 17, 2006, the disclosures of which are incorporated herein by reference in their entirety.

### FIELD

The present disclosure relates to devices that are implantable in the human body for treating obesity, in particular, morbid obesity, and very specifically the field of implants capable of artificially reducing the volume of the stomach, in particular, in order to produce a feeling of satiety in the patient.

The present disclosure relates to an expandable intragastric balloon for the treatment of obesity, comprising an external casing flexible enough to change from a first configuration in which the balloon occupies a reduced volume, called the introduction configuration, to a second configuration, called the deployed configuration, in which the balloon has its functional shape and occupies a substantially larger volume.

This present disclosure also relates to a method for producing an intragastric balloon.

### BACKGROUND

To treat obese patients, it is known to directly implant a foreign body in their stomachs, with a volume sufficient for reducing the space available for food, as well as for reducing the speed of passage of the food. These foreign bodies are, for example, implanted by the oral endoscopic approach and are generally in the form of balloons called intragastric balloons.

Such balloons normally comprise a flexible pouch made of a biocompatible elastomer material that forms the superficial casing of the balloon and that is capable of being filled, once the balloon has been implanted inside the stomach, with an inflation fluid, such as physiological serum or air.

As a result of the inflation, the balloon acquires its functional shape, i.e., a volume and a shape enabling the balloon to occupy a large part of the space normally available for food, preventing the balloon from being evacuated by the digestive tract and making the balloon substantially non-traumatic with respect to the stomach walls.

Such intragastric balloons, while offering beneficial results in terms of weight loss since they reduce the food passage kinetics and effectively help to quickly generate a feeling of satiety in the patient, nevertheless, have non-negligible disadvantages.

In particular, the removal of the balloon, necessary after a few weeks or months of implantation, which is generally done with endoscopic forceps, is a long and difficult operation for the practitioner.

Indeed, once the surgeon has deflated the balloon, i.e., tapped all or some of the fluid giving the balloon its functional shape, the flexible pouch is in the form of an entanglement of substantially shapeless material making it difficult to grasp and handle the balloon with an endoscopic instrument.

Moreover, the large amount of material to be extracted tends to cause an obstruction of the passageways through which the balloon passes when the balloon is being extracted, so that there is significant mechanical resistance to the passage of the balloon, in particular, through the esophagus, thereby increasing the risk of tearing of the flexible pouch when the balloon is gripped and pulled with the endoscopic forceps.

This extraction resistance also increases the risk of causing lesions to the natural passageways.

### SUMMARY

The present disclosure describes several exemplary embodiments of the present invention.

One aspect of the present disclosure provides an expandable intragastric balloon intended to be implanted in, then removed from, the stomach of a patient through the natural passageways, in the context of an obesity treatment, the balloon, comprising a) an external casing flexible enough to change from a first configuration in which the balloon occupies a reduced volume, called the introduction configuration, to a second configuration, called the deployed configuration, in which the balloon has its functional shape and occupies a substantially larger volume; and b) means for obtaining an extraction configuration which are capable of exerting a stress on said intragastric balloon in order to change the intragastric balloon from said deployed configuration to a third contracted configuration of a substantially smaller volume, called the extraction configuration, by acting on the external casing so that said external casing substantially returns, or tends toward, a predetermined shape corresponding to said extraction configuration.

Another aspect of the present disclosure provides a method for producing an intragastric balloon intended to be implanted in, then removed from, the stomach of a patient through the natural passageways in the context of an obesity treatment, the method comprising a) producing an external casing in which an external casing is produced that is flexible enough to change from a first configuration in which the balloon occupies a reduced volume, called the introduction configuration, to a second configuration, called the deployed configuration, in which the balloon has its functional shape and occupies a substantially larger volume; and b) moulding said casing into a shape having a shape memory effect, which tends to return said balloon from the deployed configuration to a third contracted configuration, called the extraction configuration, with a substantially smaller volume.

The invention of the present disclosure overcomes the various disadvantages mentioned above and provides a new expandable intragastric balloon for treating obesity, with a lower resistance to extraction while retaining the same functional volume and shape as the devices of the prior art when the balloon is deployed in the stomach.

One aspect of the present disclosure provides an intragastric balloon having a configuration that facilitates passage through the natural passageways, in particular, the esophagus.

Another aspect of the present disclosure provides an intragastric balloon that is easier to handle and extract.

A future aspect of the present disclosure provides an intragastric balloon that is strong enough to withstand and be mechanically protected from external stresses.

Another aspect of the present disclosure provides a method for producing an intragastric balloon intended to treat obesity that is easier to extract.

The features of the invention of the present disclosure are achieved by an expandable intragastric balloon intended to be implanted in, then removed from, the stomach of a patient through the natural passageways, in the context of an obesity treatment, in which the intragastric balloon comprises an external casing flexible enough to change from a first configuration in which the balloon occupies a reduced volume, called the introduction configuration, to a second configuration, called the deployed configuration, in which the balloon has its functional shape and occupies a substantially larger volume. The intragastric balloon comprises means for obtaining an extraction configuration, which means are capable of exerting a stress on the intragastric balloon in order to change the intragastric balloon from the deployed configuration to a third contracted configuration of a substantially smaller volume, called the extraction configuration, by acting on the external casing so that the external casing substantially returns, or tends toward, a predetermined shape corresponding to the extraction configuration.

The features of the invention of the present disclosure are also achieved by a method for producing an intragastric balloon intended to be implanted in, then removed from, the stomach of a patient through the natural passageways, as an obesity treatment, in which the production method comprises a step (a) of producing an external casing that is flexible enough to change from a first configuration in which the balloon occupies a reduced volume, called the introduction configuration, to a second configuration, called the deployed configuration, in which the balloon has its functional shape and occupies a substantially larger volume, wherein step (a) of producing the external casing includes a sub-step (b) of moulding, in which the casing is moulded into a shape that makes it possible to obtain a shape memory effect that tends to return the balloon from its deployed configuration to a third contracted configuration, called the extraction configuration, with a substantially smaller volume.

### BRIEF DESCRIPTION OF THE DRAWINGS

Other features of the invention of the present disclosure will become clearer on reading the following description, as well as the drawings, which are given purely for illustrative and non-limiting purposes

FIG. 1 shows a diagrammatic view of the extraction through the esophagus of an exemplary intragastric balloon according to the present disclosure using endoscopic forceps;

FIG. 2 shows a transverse cross-section view of an intragastric balloon according to the present disclosure in its introduction configuration;

FIG. 3 shows a transverse cross-section view of an intragastric balloon according to the present disclosure in its deployed configuration;

FIG. 4 shows a transverse cross-section view of an intragastric balloon according to the present disclosure in its extraction configuration; and

FIG. 5 shows a half-section view of an intragastric balloon according to the present disclosure in a second exemplary extraction configuration.

### DETAILED DESCRIPTION

The intragastric balloon 1 of the present disclosure is intended to be implanted in the stomach 2 of a patient in the context of an obesity treatment.

Conventionally, and as is well known to a person skilled in the art, this implantation can be performed by passing the balloon through the natural passageways, generally the esophagus. For this purpose, the balloon is usually packaged in introduction means, such as a cover, which constrict the balloon into a shape facilitating its introduction, for example, by either pulling or compressing the balloon.

Once inside the stomach 2, the balloon is removed from any packaging and is in its initial configuration, generally relaxed, called the introduction configuration, in which the balloon occupies a reduced volume.

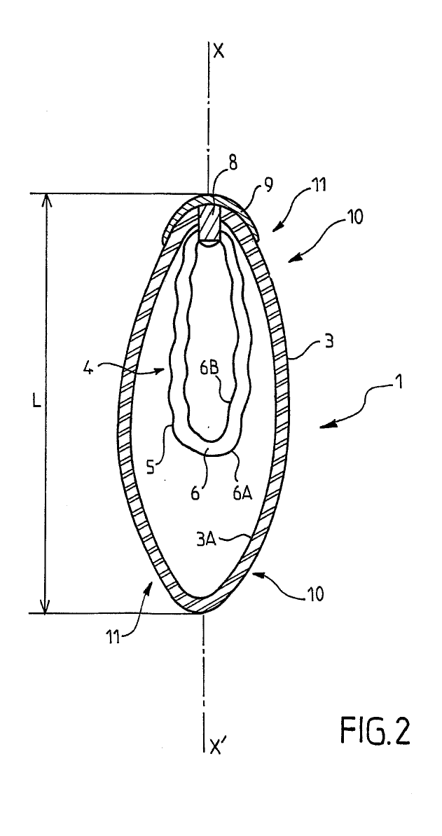

As shown in FIG. 2, the balloon I according to the present disclosure preferably has, in its introduction configuration, an ovoid shape with a substantially elliptical profile.

The practitioner then changes the intragastric balloon 1 from its introduction configuration to a second configuration, called the deployed configuration, in which the balloon has its functional shape and occupies a substantially larger volume.

For purposes of the present disclosure, the terms referring to the various configurations, such as “introduction configuration” and “deployed configuration”, can be applied indifferently to the balloon 1 and to the parts constituting the balloon, discussed in the corresponding art.

For purposes of the present disclosure, the term “functional shape” refers to the volume and to the shape conferred on the balloon in order to give the balloon the therapeutic capacity intended in the context of an obesity treatment.

In particular, the balloon 1 should normally occupy a non-negligible volume in the gastric cavity so as to create a rapid feeling of satiety in the patient accompanied by a decrease in the amount of food ingested. In addition, the geometric, physical and chemical properties of the balloon 1 should help to minimize the traumatic impact that the balloon has on the stomach walls.

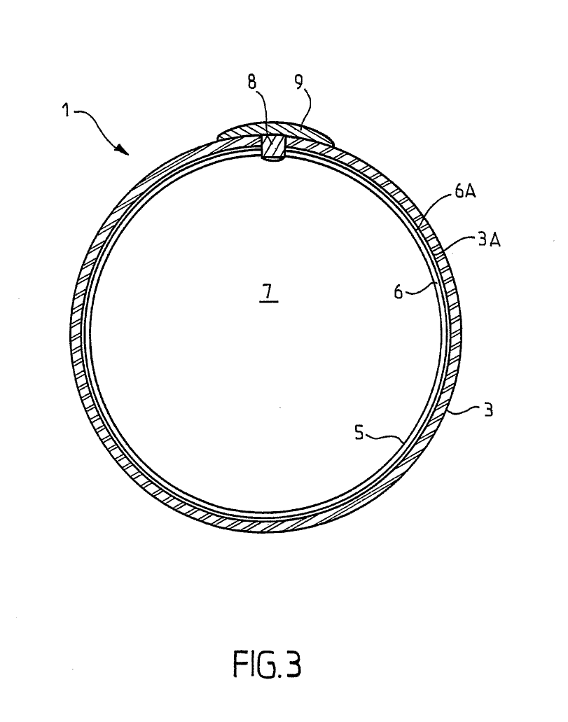

According to one exemplary embodiment of the present disclosure, the balloon is designed to adopt a substantially spherical shape in its deployed configuration, as shown in FIG. 3.

To this end, the intragastric balloon 1 intended for obesity treatment is expandable and has an external casing 3 flexible enough to change from a first configuration in which the balloon occupies a reduced volume, called the introduction configuration, to a second configuration, called the deployed configuration, in which the balloon has its functional shape and occupies a substantially larger volume.

Thus, the external casing 3 is preferably made of a flexible elastomer material, for example, silicone. Of course, the material constituting the external casing 3 may be subject to alternative compositions, in particular, treatments intended to modify its properties, such as transparency, colour, imperviousness or elasticity, without going beyond the scope of the invention.

Advantageously, the balloon 1 also has shaping means 4 capable of causing the balloon to change from its introduction configuration to its deployed configuration. Preferably, the shaping means 4 include an inflation pouch 5.

For purposes of the present disclosure, the term “inflation pouch” refers to an inflatable member that has a membrane 6 delimiting an internal substantially variable volume which is mounted inside the external casing 3 and which is capable of being filled with an inflation fluid 7. Thus, the inflation pouch 5 is intended to receive a fluid 7, and more specifically to contain the fluid in a sealed chamber.

To enable the inflation pouch 5 to be connected to pump or reservoir-type filling means, the intragastric balloon 1 also has connection means, preferably formed by an inflation valve 8, for example, made of a self-closing membrane. According to one exemplary embodiment, the valve 8 is assembled with the inflation pouch 5 and with the external casing 3.

According to the present disclosure, as is shown in the figures, the inflation pouch 5 is primarily, or even entirely, distinct from the external casing 3 to which the inflation pouch may be mechanically attached at the level of the inflation valve 8. The arrangement of the external casing/inflation pouch can thus be similar to a tire/air chamber assembly.

The balloon 1 can also comprise additional attachment and/or reinforcement elements 9, for example, in the form of flexible discs.

To cause the balloon to change from its introduction configuration to its deployed configuration, the practitioner injects an inflation fluid into the pouch 5 through the valve 8, for example, by means of a cannula, so that the expansion of the pouch 5 causes the pouch to exert stress on the external casing 3 thereby tending to push the external casing in substantially centrifugal directions.

Preferably, the inflation pouch 5 and the external casing 3 are substantially separate. The transmission of the motor stress of the inflation pouch 5 onto the external casing 3 occurs when the external surface 6A of the membrane 6 substantially comes into contact with the internal surface 3A of the external casing.

As shown in FIG. 3, the external casing 3 preferably substantially matches the shape of the inflation pouch 5 in the deployed configuration.

Preferably, the inflation pouch 5, before inflation, has overall dimensions substantially smaller than those of the external casing 3.

Thus, to push the external casing 3 by means of the inflation pouch 5, it is necessary to increase the volume of the pouch 5 and to distend the membrane 6 by inflation.

To this end, the membrane 6 is preferably ductile and has a mechanical deformation resistance low enough to enable the membrane to distend when pressurised inflation fluid is injected.

According to an important feature of the present disclosure, the ratio between the surface, in particular, external, of the inflation pouch 5 in the deployed configuration and the surface thereof in the introduction configuration is such that the change from the introduction configuration to the deployed configuration causes a residual plastic deformation of the pouch 5. For purposes of the present disclosure, the term “residual plastic deformation” means a deformation capable of remaining at least partially after removal of the inflation stress, in particular, when the balloon is emptied.

Thus, as shown in FIG. 3, the volume of the inflation pouch 5 considered in the deployed configuration can be substantially greater than the initial volume of the inflation pouch, and preferably substantially equal to the total volume occupied by the balloon 1 in the stomach 2.

In particular, the ratio between the surface of the inflation pouch 5, and/or the membrane 6, in the deployed configuration and the surface of the pouch, respectively of the membrane, in the introduction configuration, can be substantially between 2 and 5, and preferably substantially equal to 3.3.

When it is necessary to extract the intragastric balloon 1, i.e., when it needs to be removed from the stomach 2 preferably through the natural passageways, in particular, the esophagus, it is necessary to deflate the balloon by tapping the inflation fluid 7 contained in the pouch 5. To this end, a needle-type endoscopic instrument can be inserted into the balloon 1 by perforation through the casing 3 and the membrane 6, at any point of the balloon.

When the stress holding the balloon in its deployed configuration, i.e., in its functional shape, is relaxed, the balloon returns, according to an important feature of the present disclosure, to a shape conducive to its collection in the stomach and its extraction through the natural passageways using endoscopic forceps 20.

To this end, the intragastric balloon 1 according to the present disclosure has means for obtaining the extraction configuration 10 capable of causing the balloon to change from the deployed configuration to a third contracted configuration, called the extraction configuration, with a volume substantially smaller than that which the balloon occupied in the deployed configuration, in order to facilitate its extraction through the natural passageways.

More specifically, the means for obtaining an extraction configuration 10 are capable of exerting a stress on the intragastric balloon 1 in order to cause the balloon to change from the deployed configuration to the extraction configuration by acting on the external casing 3 so that the external casing 3 is substantially, or tends toward, a predetermined shape corresponding to the extraction configuration.

Advantageously, the means for obtaining the extraction configuration 10 prevent the balloon, by deflating, from forming a randomly distributed, disordered mass of material capable, in particular, of forming thick folds making extraction difficult.

In other words, the means for obtaining the extraction configuration exert a positive action on the balloon when the balloon is being deflated, and more specifically on the external casing 3, so as to substantially predictably organize the material forming the external casing, and more generally the balloon, in an arrangement conducive to its collection and extraction by endoscopy.

The extraction configuration therefore corresponds to a state of the balloon in which the balloon has not only a relatively small volume, but also a particular arrangement of its constituent material.

To enable the intragastric balloon 1 to move, preferably spontaneously, into the extraction configuration when the stress holding the balloon in the deployed configuration is relaxed, the means for obtaining the extraction configuration 10 can include pseudo-elastic return means 11, which are stressed when the external casing 3 is in the deployed configuration and which tend to contract the balloon toward its extraction configuration.

For purposes of the present disclosure, the term “pseudo-elastic” describes return elements 11 that are capable of being deformed and/or of deforming the balloon 1, but the behaviour of which is not necessarily strictly elastic, or entirely reversible, or absolutely deterministic. In other words, the pseudo-elastic return elements 11 can, in particular, induce a simple tendency to bend or to constrict, without causing the balloon to adopt an extraction configuration that is, for example, strictly identical to its introduction configuration. Moreover, the return elements 1 are not necessarily in a resting state free of any mechanical stress, in particular, internal, when the balloon is in the extraction configuration.

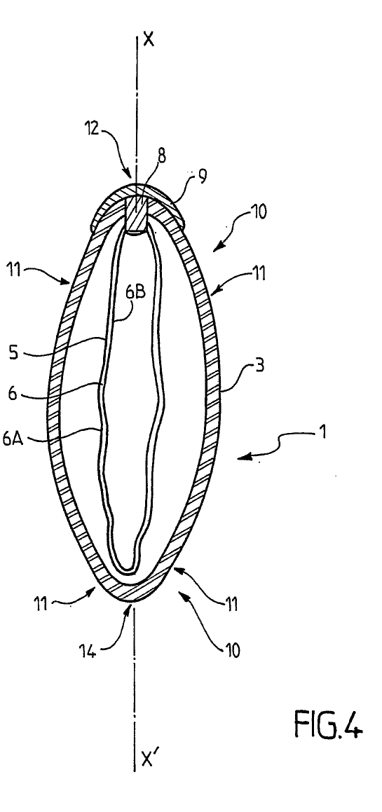

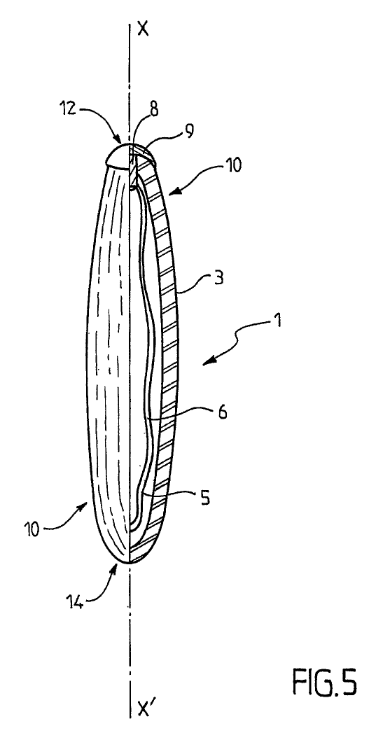

As shown in FIGS. 4 and 5, the means for obtaining the extraction configuration 10 can, in particular, be capable of acting on the external casing 3 in order to give the external casing a substantially elongate shape.

Advantageously, the means for obtaining the extraction configuration 10 are also capable of facilitating the emptying of the balloon 1 by exerting a stress on the balloon and, in particular, on the inflation pouch 5 which tends to push the inflation fluid outside the balloon, helping, for example, to flush the fluid through the cannula used by the practitioner to perform the puncture.

The means for obtaining the extraction configuration 10 and the shaping means 4 can, in particular, be substantially antagonistic.

According to an exemplary embodiment of the present disclosure, the means for obtaining the extraction configuration 10 are formed by the external casing 3, and the pseudo-elastic return elements 11 are formed by the membrane structure. Thus, the external casing 3 has an intrinsic shape memory, preferably conferred by its constituent material, which tends to return the external casing spontaneously from its deployed configuration to its extraction configuration.

For purposes of the present disclosure, the term “shape memory” means the capacity to substantially find, or tend toward, a shape or dimension that has been predetermined, for example, by a sizing and/or a particular production process.

Thus, according to an exemplary embodiment, the external casing 3 can be made at least partially of a silicone-type elastomer material.

Preferably, the silicone casing will have a substantially ovoid shape, and even more preferably a substantially elliptical shape. However, other formats can, of course, be adopted, for example, pear-shaped pouches, or even multi-lobe pouches, without going beyond the scope of the present disclosure.

Thus, the means for obtaining the extraction configuration 10 are preferably arranged so as to confer on the external casing 3, when the external casing is in the extraction configuration, a substantially streamlined shape with two apexes 12, 14.

According to an exemplary embodiment not shown, the return elements 11 can be formed, for example, by ribs secured to the external casing 3 and arranged in an umbrella manner around the apexes of the streamlined shape, or by constriction rings, or by one or more spiral springs. The return elements can, in particular, form a sort of substantially ovoid reinforcement or cage matching the shape of the external casing 3.

Depending on the means used to obtain the extraction configuration 10, and depending on the severity of the stress exerted on the balloon 1 in order to return the balloon to the extraction configuration, the balloon 1 may have a more or less contracted shape, and in particular, a more or less flattened elliptical shape, in the extraction configuration.

According to a particularly advantageous structural arrangement, the valve 8 can be arranged at the level of one of the apexes 12, 14, in the extension of the streamlined shape. More generally, the most rigid and/or the thickest areas of the balloon, such as those comprising valves, elements offering a reinforced grip, reinforcing elements or junction elements, can advantageously be located at the level of the apexes 12, 14 of the streamlined shape.

Thus, the arrangement of the material constituting the balloon in the extraction configuration advantageously tends to cause the most mechanically resistant elements or areas, i.e., those more capable of being grasped and handled, to emerge. This accessibility of the areas conducive to grasping, and more specifically the most rigid and/or the thickest elements such as the inflation valve or the attachment and/or reinforcement element, also makes it easier to locate and grasp the areas by endoscopy.

For purposes of the present disclosure, the term material constituting the balloon means, in a non-limiting manner, all of the various elements constituting the balloon 1, such as the external casing 3, the inflation pouch 5, the valves 8 or the other attachment and/or reinforcement elements 9, considered without distinction as a general mass of material having a volume and a shape that are variable, in particular, according to the presence of inflation fluid 7 or inflation fluid residue.

In addition, the substantially thick and/or rigid elements used in the composition of the balloon 1, such as the inflation valve 8 and attachment and/or reinforcing elements 9, will preferably be located in the longitudinal extension of the balloon 1, i.e., oriented so that the elements have, in a cross-section normal to the longitudinal axis (XX′) of the balloon, in the extraction configuration, their smallest dimension and/or their lowest mechanical resistance.

In particular, the rigid or thick elements used in the composition of the balloon 1, such as the valves 8, will preferably be located at the level of the apexes 12, 14 of the external casing 3, and thus are distributed and ordered substantially according to a preferred axis of extension (XX′) in the extraction configuration.

Thus, the rigid elements will be capable of being engaged in a line in the natural passageways when the balloon 1 is extracted and moved through the esophagus 17.

The alignment of the rigid elements in the direction of movement will limit the risk of the elements catching the walls of the esophagus and of the walls presenting excessive resistance to the movement of the balloon.

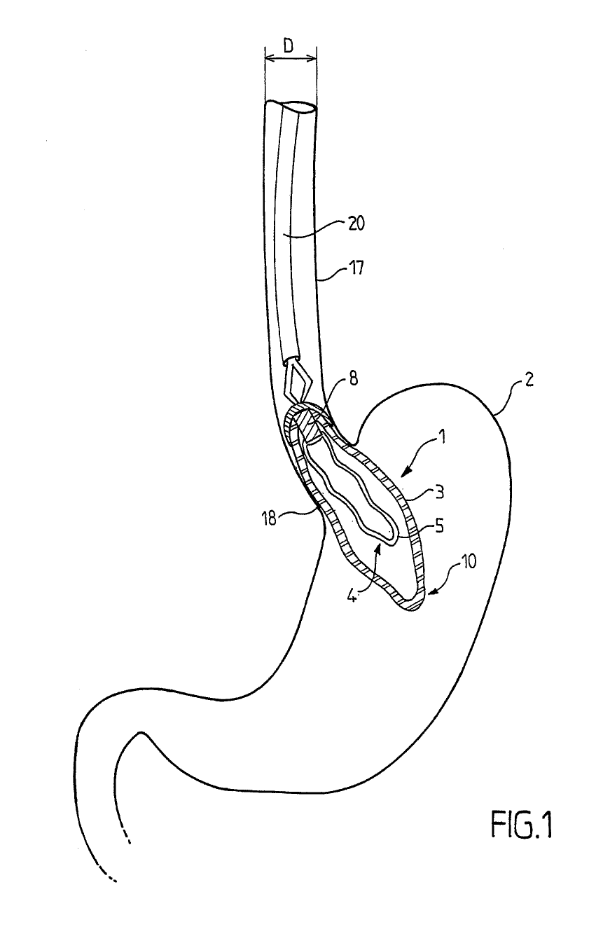

In a particularly advantageous manner, the change toward the extraction configuration has the effect of reorganizing the arrangement of the material constituting the balloon, of distributing it so that it appears as an access means, facilitating its frontal engagement in the esophagus 17, then its movement in a line through it, as shown in FIG. 1.

In particular, the substantially pointed streamlined shape of the balloon 1 in the extraction configuration enables a progressive engagement of cross-sections of increasing diameter of the balloon in the esophagus 17, thereby facilitating the crushing and the regular distribution of the material constituting the balloon, in particular, at the level of the neck created by the cardia 18.

Moreover to prevent engorgement of the natural extraction passageways due, for example, to the formation of a fold or a mass of material constituting the balloon 1, in particular, when the balloon must pass through a segment of the passageways of which the cross-section narrows to form a funnel capable of causing a blockage, the shape adopted by the balloon 1 in the extraction configuration tends, according to an important feature of the present disclosure, to minimize the total thickness of the constituent material in each cross-section normal to the longitudinal axis (XX′) of the balloon. In other words, the material constituting the balloon 1 in the extraction configuration is capable of passing without being jammed through any esophageal segment of the patient into the stomach, which has been implanted with the balloon 1, in particular, through a substantially tubular segment with a diameter D of around 18 mm.

To this end, and according to a feature that can constitute an entirely separate invention, independently of the presence of means for obtaining the extraction configuration, the inflation pouch 5 is capable of being substantially uniformly plastically distended by the inflation fluid 7, during the change from the introduction configuration to the deployed configuration, so as to be capable of having, in the extraction configuration, i.e., after the stress has been relaxed and the fluid has been emptied, a thickness of its membrane 6 substantially lower than in the introduction configuration.

As shown in FIG. 4 and FIG. 5, the inflation pouch 5 can retain residual plastic deformation in the extraction configuration that involves sizing modifications thereof.

Preferably, the inflation pouch 5 has a substantially more elongate shape and a more extended perimeter than in the introduction configuration, and consequently a lower thickness over the entire length. In other words, the material constituting the membrane 6 is rearranged by plastic deformation, preferably so that the membrane 6 is finer and its internal 6B and external 6A surfaces are more extended in the extraction configuration than they were in the introduction configuration.

Thus, the total of the cumulative material thicknesses of the inflation pouch 5 and the external casing 3, measured in a direction orthogonal to the longitudinal axis (XX′) of the balloon 1, is substantially lower in the extraction configuration than in the introduction configuration.

In addition, and particularly advantageously, this reduction in thickness of the material constituting the inflation pouch 5 enables the means for obtaining the extraction configuration 10 to act more effectively, in particular, when the means crush or force a portion of the material constituting the balloon. In particular, the inflation pouch 5 has, after plastic deformation, a mechanical resistance to deformation, in particular, to bending or compression, lower than what the resistance would have been if the membrane 6 had been thicker, in particular, substantially as thick as in the introduction configuration. In other words, the stretching of the inflation pouch 5 resulting from the distension thereof tends to facilitate the folding of the pouch by the means for obtaining the extraction configuration 10.

Finally, according to an exemplary embodiment shown in FIG. 5, it is even possible to consider equipping the balloon 1 with means for obtaining the extraction configuration 10 that are arranged and sized so that the folding of the external casing 3 on itself and the crushing of the inflation pouch 5 resulting therefrom are such that the balloon 1 is in a substantially compacted shape, as if the balloon were constricted.

One exemplary method for producing an intragastric balloon 1 according to the present disclosure will now be described.

One method for producing an intragastric balloon intended for the treatment of obesity comprises a step (a) of producing an external casing, in which an external casing 3 is produced that is flexible enough to change from a first configuration in which the balloon occupies a reduced volume, called the introduction configuration, to a second configuration, called the deployed configuration, in which the balloon has its functional shape and occupies a substantially larger volume.

According to an exemplary embodiment, step (a) of producing an external casing includes a sub-step (b) of moulding the casing into a shape that makes it possible to obtain a shape memory effect, which tends to return the balloon from its deployed configuration to a third contracted configuration, called the extraction configuration, with a substantially smaller volume.

Preferably, the external casing 3 is produced by injection moulding a silicone-type elastomer. Advantageously, the features of the injection mould, such as the shape and the distribution of thicknesses of the imprint, will be chosen so as to give the external casing 3 shape memory.

Thus, according to an exemplary embodiment, the sub-step (b) of moulding includes the production of an external casing 3 of which the shape is substantially elongate, and, even more preferably, streamlined with two apexes 12 and 14.

In particular, an external casing 3 moulded into an ovoid shape with a substantially elliptical sagittal cross-section will give the balloon a tendency to fold onto itself, at least partially, when the inflation stress is relaxed, with the apexes 12, 14 of the ellipse then withdrawing somewhat like a closing umbrella.

Advantageously, the external casing 3 can have, at rest, a variable thickness, for example, a finer median area combined with thicker apexes, or the reverse. By adapting the thickness of its membrane structure, it is especially possible to envisage controlling the deformation of the casing 3 in preferred directions during inflation or to reinforce the return effect toward the extraction configurations.

The method for producing the balloon 1 also preferably includes a step (c) of adding to the external casing 3 an impervious inflation pouch 5 capable of being plastically distended when filled with a pressurised fluid 7.

To satisfy the requirements of imperviousness and ductility specific to its function, the inflation pouch 5 is preferably made of a polymer with a gas permeability lower than that of silicone, for example, thermoplastic polyurethane.

The inflation pouch 5 can advantageously be made by joining at least two polyurethane sheets, for example, by welding or bonding along a peripheral welding strip or line.

Preferably, each sheet can be formed by at least two superimposed polyurethane films, which films may or may not be secured together, for example, by bonding. This stratified structure makes it possible, in particular, to easily adjust the material thicknesses and improve the imperviousness.

According to another exemplary embodiment, the junction between the sheets has a substantially circular shape. In addition, the polyurethane sheets can be pre-shaped during a shaping step (d), for example, by thermoforming or preferably by drawing, so as to give the polyurethane sheets a hemispheric shape. Particularly advantageously, the inflation pouch 5 thus has a substantially spherical shape.

Advantageously, the inflation pouch 5 is preferably arranged so that the pouch substantially retains its spherical shape as the pouch expands, and the thinning of the membrane 6 is substantially uniform.

For purposes of the present disclosure, the term “substantially uniform” means that the pull on the material created by the distension of the pouch 5 is distributed substantially uniformly throughout the membrane 6, thereby resulting in a thinning that is almost regularly distributed.

Thus, according to an important feature of the present disclosure, the membrane 6 of the inflation pouch 5 can be distended during the inflation without having excessive local necking phenomena capable of significantly weakening the membrane or causing the membrane to break, in particular, by tearing or bursting. The inflation pouch 5 according to the present disclosure is therefore preferably particularly strong.

Moreover, the inflation pouch 5 is also designed to retain its properties of imperviousness to the inflation fluid, even after the inflation pouch has been distended.

Thus, preferably, during step (c) of adding the inflation pouch, a sub-step (c′) of sizing the inflation pouch 5, in particular, with respect to the external casing 2, as well as a step (c″) of selecting the materials constituting its membrane 6, will be carried out, so that the deformation of the inflation pouch 5 during the change from the introduction configuration to the deployed configuration is essentially plastic, and the elastic return of its membrane 6 during the change from the deployed configuration to the extraction configuration is low, and even substantially negligible.

Thus, according to an important feature of the present disclosure, the pair of parameters (material and size with respect to the external casing 3) of the inflation pouch 5 is determined so that it satisfies at least two requirements: the first being that the pouch can be deformed without being significantly weakened or losing its imperviousness combined with a sufficient volume for conferring on the external casing with which the pouch is engaged its functional shape in the deployed configuration, and the second being that the deformation necessary for arriving at the deployed configuration causes a plastic deformation of the membrane 6 so that, after the stress has been relaxed, when the balloon is in the extraction configuration, a residual plastic distension remains.

The method for producing a balloon 1 according to the present disclosure can also include a step (e) of mounting at least one valve 8, through which fluid transfer systems can be introduced, such as cannulae. During this step (e) of mounting the valve, the valve 8 is preferably placed substantially in the extension of the streamlined shape conferred by the moulding on the external casing 3, even more preferably at the level of one of the apexes 12, 14 thereof.

Advantageously, the substantially thick and/or rigid elements used in the composition of the balloon 1 will preferably be placed in the longitudinal extension of the balloon 1, i.e., oriented so that the substantially thick and/or rigid elements have, in a cross-section normal to the longitudinal axis (XX′), their smallest dimension and/or their lowest mechanical resistance. In particular, preferably, structural arrangements contributing to an alignment of the rigid or thick elements in the predicted direction of extraction of the balloon 1 will be preferred so as to limit the areas capable of being caught or of leading to an accidental catching or an adhesion to the walls of the extraction passageways.

According to a preferred and non-limiting exemplary embodiment, the sizes of the various aforementioned element can be determined as follows:

The external casing 3, at rest, in particular, in the introduction configuration and/or after the moulding step (b), can have an ovoid or ellipsoid shape of which the sagittal cross-section corresponds substantially to an ellipse with a large axis L of around 100 mm and a small axis of around 85 mm.

The inflation pouch 5 can be made by joining two sheets each formed by two films with a thickness of 100 μm, preformed by drawing and welded according to a circular profile around 70 mm in diameter. A deflated pouch is thus obtained, of which the membrane 6 has a thickness of around 200 μm in the introduction configuration.

In the deployed configuration, the balloon 1 can then occupy a functional volume of around 700 cm³, substantially in the shape of a sphere with a diameter of approximately 100 mm to 110 mm.

Preferably, the volume of the inflation pouch 5 in the deployed configuration will correspond substantially to the functional volume of the balloon 1, i.e., around 700 cm³.

In the extraction configuration, the residual plastic distension of the inflation pouch 5 can, in this case, cause a reduction in the thickness of the membrane 6, with the thickness after distension being substantially between 50 and 70 μm.

It should be noted that, even though the observed reduction in thickness is substantially uniform, the initial thickness of the membrane 6 is capable of being approximately preserved in the immediate vicinity of the weld line that joins the two polyurethane sheets. In practice, this residual over-thickness can be in the form of a wide strip of around 1 to 2 mm extending on each side of the junction line.

It is possible to envisage alternative embodiments of a balloon 1 with shape memory according to the present disclosure comprising a single pouch, instead of the assembly constituted by the inflation pouch 5 and the external casing 3, just as it is possible to produce pouches and/or casings with a plurality of lobes, for example, substantially in the shape of orange quarters joined at their apexes, without going beyond the scope of the invention.

Thus, the intragastric balloon 1 according to the present disclosure advantageously makes it possible to spontaneously optimize the distribution of the material inside the patient's stomach, in particular, at the end of the treatment, thus reducing the amount of material to be frontally engaged in the esophagus 17.

The preferred line presentation, according to an elongate shape with regular, substantially non-traumatic contours, of the material constituting the balloon 1 advantageously facilitates the insertion of the balloon from the stomach 2 into the esophagus 17, then the passage of the balloon through the esophagus.

In a particularly advantageous manner, the folding and preferably ovalisation effect obtained by the shape memory conferred on the external casing 3 is facilitated by the thinning of the membrane 6, which then has a lower resistance to deformation and bending.

In addition, the overall relaxation of the structure of the balloon 1 obtained, in particular, by the residual plastic distension of the inflation pouch 5 facilitates the engagement and movement of the balloon through the esophagus 17.

1. An expandable intragastric balloon for implantation in, and removal from, the stomach of a patient through the natural passageways, in the context of an obesity treatment, the balloon, comprising:
a) an external casing having sufficient flexibility to change from a first configuration in which the balloon occupies a reduced volume, defined as an introduction configuration, to a second configuration, defined as a deployed configuration, in which the balloon has a functional shape and occupies a substantially larger volume than when in the introduction configuration; and
b) means for configuring said balloon into a volume smaller than said deployed configuration which means are capable of exerting a stress on said intragastric balloon in order to change the intragastric balloon from said deployed configuration to a third contracted configuration of a substantially smaller volume, defined as extraction configuration, by acting on the external casing so that said external casing substantially returns, or tends toward, a predetermined shape corresponding to said extraction configuration.

2. The intragastric balloon of claim 1, wherein the means for obtaining the extraction configuration are capable of acting on the external casing in order to form the external casing into a substantially elongate shape.

3. The intragastric balloon of claim 2, wherein the substantially thick or rigid elements used in the composition of the balloon, such as the inflation valve and attachment or reinforcing elements, are oriented so that the substantially thick or rigid elements have, in a cross-section normal to the longitudinal axis of said balloon, either the smallest dimension or the lowest mechanical resistance.

4. The intragastric balloon of claim 1, wherein the arrangement of the material constituting said intragastric balloon in the extraction configuration tends to cause the most rigid or thickest elements of said balloon, such as the inflation valve and the attachment or reinforcement elements, to emerge to make it easier to locate and grasp the most rigid or thickest elements of said balloon by endoscopy.

5. The intragastric balloon of claim 1, wherein the means for obtaining the extraction configuration comprises pseudo-elastic return elements which are stressed when the external casing is in the deployed configuration and which are biased to contract the intragastric balloon toward the extraction configuration.

6. The intragastric balloon of claim 1, wherein the means for obtaining the extraction configuration are arranged to form the external casing, when it is in the extraction configuration, into a substantially streamlined shape with two apexes.

7. The intragastric balloon of claim 6, wherein the rigid or thick elements used in the composition of the balloon are located at the level of the apexes of the external casing.

8. The intragastric balloon of claim 1, wherein the means for obtaining the extraction configuration are formed by the external casing.

9. The intragastric balloon of claim 1, wherein the external casing is made of silicone.

10. The intragastric balloon of claim 1, further comprising shaping means capable of causing said balloon to change from the introduction configuration to the deployed configuration, which means include an inflation pouch for receiving a fluid.

11. The intragastric balloon of claim 10, wherein the ratio between the surface of the inflation pouch in the deployed configuration and said surface in the introduction configuration is between 2 and 5.

12. The intragastric balloon of claim 10, wherein the ratio between the surface of the inflation pouch in the deployed configuration and said surface in the introduction configuration is such that the change from the introduction configuration to the deployed configuration causes a residual plastic deformation of said pouch.

13. The intragastric balloon of claim 10, wherein the inflation pouch is substantially uniformly plastically distended by the inflation fluid, so as to be capable of having, in the extraction configuration, a thickness of its membrane substantially lower than in the introduction configuration.

14. A method for producing an intragastric balloon for implantation in, and removal from, the stomach of a patient through the natural passageways in the context of an obesity treatment, the method comprising:
a) producing an external casing in which an external casing is produced that is flexible enough to change from a first configuration in which the balloon occupies a reduced volume, defined as an introduction configuration, to a second configuration, defined as a deployed configuration, in which the balloon has its functional shape and occupies a substantially larger volume; and
b) moulding said casing into a shape having a shape memory effect, which tends to return said balloon from the deployed configuration to a third contracted configuration, defined as an extraction configuration, with a substantially smaller volume than the deployed configuration.

15. The method of claim 14, wherein the moulding step b) further comprises a sub-step b′) producing an external casing having a substantially elongate shape.

16. The method of claim 14, further comprising a step c):
c) adding an impervious inflation pouch to said external casing, wherein the impervious inflation pouch can be plastically distended when filled with a pressurized fluid.

17. The method of claim 15, further comprising a step e):
e) mounting at least one valve wherein said valve is placed substantially in the extension of the elongate shape.

18. The intragastric balloon of claim 10, wherein the ratio between the surface of the inflation pouch in the deployed configuration and said surface in the introduction configuration is substantially equal to 3.3.

19. The intragastric balloon of claim 11, wherein the ratio between the surface of the inflation pouch in the deployed configuration and said surface in the introduction configuration is such that the change from the introduction configuration to the deployed configuration causes a residual plastic deformation of said pouch.

20. The method of claim 15, wherein said external casing is streamlined and has two apexes.

21. The method of claim 16, further comprising a step d):
d) shaping said polyurethane sheets into a hemispherical shape.
